(19) United States
(12) Patent Application Publication
(10) Pub. No.: US 2024/0307509 A1
(43) Pub. Date: Sep. 19, 2024

(54) PLACENTAL ALKALINE PHOSPHATASE FOR STARTING EARLY TREATMENT OF CANCER PATIENTS EXHIBITING NO DETECTABLE LOSS OF BODY AND MUSCLE WEIGHT AS YET TO PREVENT OR REDUCE THE LOSS OF MUSCLE PROTEINS

(71) Applicant: MNPHARM SBC, White Bear Lake, MN (US)

(72) Inventor: Zoltan Kiss, Austin, MN (US)

(21) Appl. No.: 18/270,349

(22) PCT Filed: Jan. 6, 2021
(86) PCT No.: PCT/US2021/012273

§ 371 Date: Jun. 29, 2023

(51) Int. Cl. A61K 38/46; A61P 35/00

(52) U.S. Cl. CPC A61K 38/465; A61P 35/00; C12Y 301/03001

(57) ABSTRACT

This invention provides placental alkaline phosphatase or an active derivative thereof as an adjuvant early treatment of chemotherapy treated cancer patients to reduce or prevent the loss of skeletal muscle proteins. Consequently. administration of placental alkaline phosphatase at early phases of tumor development will enhance the efficacy of chemotherapy eventually leading to increased life expectancy of the cancer patient.

### FIELD OF INVENTION

This invention provides placental alkaline phosphatase as part of the chemotherapy regimen for early start of treatment of cancer patients, who at that time still maintain normal body and muscle weight, to prevent or reduce loss of skeletal muscle proteins and thus enhance the efficacy of cancer therapies and enhance life expectancy.

### BACKGROUND

### The Consequences of Loss of Muscle Proteins in Cancer Patients.

In the present clinical practice, the combination of weight loss as well as the loss of muscle mass and muscle strength is called cancer cachexia which is said to be the direct cause of about 20-30% of cancer deaths, while also representing a significant contributing factor to most additional cancer deaths [Aoyagi, T., Terracina, K.P., Raza, A., Matsubara, II. and Takabe, K. Cancer cachexia, mechanism, and treatment. World J. Gastrointest. Oncol. 7, 17-29, 2015]. In most cases, a cachectic patient, as the disease progresses, will have less appetite, less vitality, and less ability to tolerate cancer treatments.

Tumor and chemotherapy induced substantial loss of muscle proteins is the direct cause of muscle weakness that can occur even in the absence of noticeable weight loss. Cancer patients with significant muscle depletion are more prone to severe drug-associated toxicity and show a poorer prognosis overall. Currently there is no effective treatment to prevent loss of muscle proteins even when it is recognized. However, the ratio of muscle proteins to the total muscle mass (which is about 70-75% water) is relatively small. Taking this into account and the fact that changes in muscle proteins greater than 30% already lead to death, health care providers usually miss relatively small, but physiologically still important, reductions in muscle proteins. This present situation calls for (a) a rapid, reliable, and economical test to determine if the cancer patients already started to lose significant amounts of muscle proteins, and (b) for an early effective treatment that can keep muscle protein loss at bay. Early intervention is important because beyond certain level the loss of protein cannot be reversed or even stabilized.

Tumor development is associated with a type of muscle damage resulting in the activation of both satellite and non-satellite muscle progenitor cells. Activated satellite cells (myotubes) are normally committed to a myogenic program but are inhibited by tumor derived signal(s) from completing differentiation by persistent expression of the self-renewing factor Pax7 [Wei A. H., Berardi, E., Cardillo, M. V. et al. NF-KB-mediated Pax7 dysregulation in the muscle microenvironment promotes cancer cachexia. J Clin Invest. 123, 4821-4835, 2013]. The non-satellite cell population is a heterogeneous group of mesenchymal stem cells (MSCs) that includes interstitial cells called PW1+/Pax7− Interstitial Cells), fibroadipogenic progenitors, muscle side population cells, and muscle resident pericytes. Under certain conditions, MSCs can develop myogenic properties which can help muscle regeneration [Testa1. S., Rieral, S.C., Fornetti, E. et al. Skeletal muscle-derived Human Mesenchymal Stem Cells: influence of different culture conditions on proliferative and myogenic capabilities. bioRxiv preprint doi: https://doi.org/10.1101/2020.05.12.090746; posted on May 13, 2020].

The most often used criteria for diagnosing cachexia in cancer patients, determined by a panel of experts, is weight loss greater than 5% over the past 6 months (in the absence of voluntary starvation). Serious body weight loss (greater than 5%) that is most often associated with muscle loss and weakness presently cannot be reversed by any known method (medication, diet, appetizers). However, cachexia may be prevented by early treatment of the cancer patient with an appropriate drug when the loss of body weight is still less than 2%.

In the clinical practice, treatment of cancer cachexia with appetite enhancers and other means (that are not sufficiently effective in the first place) usually starts too late to have a significant impact. Some drugs, such as megestrol acetate (a synthetic derivative of progesterone) have been in the clinical practice but show only modest results. Some other drugs had been or still are in the clinical trial stage (drugs blocking myostatin, activin, or the activin receptor ActRIIB, etc), or failed to receive approval (Osterine, inhibitor of ActRIIB, etc.).

### Cyclophosphamide (Cyp) as the Model Chemotherapeutic Drug for Cancer Treatment in this Invention.

In this invention, Cyp, marketed as Cytoxan or Neosar, is used as the model anticancer compound (usually used in combination with other anticancer drugs) to induce loss of skeletal muscle proteins. Its chemical name is 2-[Bis (2-chloroethyl) amino] tetrahydro-2H-1, 3, 2-oxazaphosphorine 2-oxide monohydrate; its molecular formula is C₂H₁₅Cl₂N₂O₂P and its molecular weight is 261.1.

Cyp is an inactive drug. However, through the action of the cytochrome P-450 oxidase system in the liver, Cyp is converted into phosphor-amide mustard and acrolein. It is the phosphor-amide mustard which introduces alkyl radicals (CₙH₂ₙ₊₁) at the number 7 nitrogen atom of the guanine base in the DNA strands thereby forming DNA cross-linkage. Cross-linked cancer cell DNA is unable to replicate and complete normal cell division. As a result, cancer cells stop proliferating, causing them to die by apoptotic cell death.

Cyp has been in use for more than 40 years to treat breast cancer, testicular, endometrial, ovarian, hormone dependent prostate, and lung cancers, neuroblastoma, retinoblastoma, rhabdomyosarcoma and Ewing's sarcoma, Hodgkin's and non-Hodgkin's lymphoma, Burkitt's lymphoma, chronic lymphocytic leukemia, chronic myelocytic leukemia, acute myelocytic leukemia, acute lymphocytic leukemia, t-cell lymphoma, and multiple myeloma.

### SUMMARY OF THE INVENTION

This invention provides highly purified human placenta-derived placental alkaline phosphatase (PLAP), or a recombinant active derivative of PLAP (rPLAP), for the adjuvant treatment of cancer patients on chemotherapy soon after tumor detection. The goal is to prevent or reduce loss of muscle proteins. At early stages of tumor development, no obvious loss of body and muscle weight occurs while skeletal muscle starts losing proteins which is further exacerbated by chemotherapy. It is critical to start implementing a PLAP containing chemotherapy regimen as early as possible to prevent or reduce the loss of muscle proteins both at the earliest and later stages of tumor development. This is important, because if the loss of muscle proteins reaches a critical level, and parallel to that the cancer patient also start losing body and muscle weight entering the phase of advanced cachexia stage (substantial simultaneous loss of body weight, muscle mass, and muscle proteins), from that point on cachexia cannot be controlled well and certainly cannot be reversed. In this disclosure and claims the phrase “cancer patient without cachexia” means a cancer patient that presented to the clinic with less than 2 percent involuntary loss of body weight during a 25 day period preceding tumor detection.

Injected PLAP with a long (7 to 10 days) half-life time in the circulation will be used. While PLAP is added to the chemotherapy as an adjuvant at the earliest time possible after tumor detection, PLAP will remain part of the chemotherapy as far as needed.

In one embodiment, this invention provides PLAP as part of chemotherapy regimens for early start of treatment of cancer patients with normal weight to prevent or reduce early loss of skeletal muscle proteins induced by the tumor and/or chemotherapy.

In a second embodiment, PLAP is used to enhance the efficacy of chemotherapy on reducing tumor growth.

In a third embodiment, PLAP is used to enhance life expectancy of chemotherapy treated cancer patients as a result, at least in part, of early intervention to prevent or reduce loss of muscle proteins thereby allowing more efficient treatments.

In a fourth embodiment, PLAP is used to reverse tumor induced reduction of serum triglyceride, a marker of early loss of skeletal muscle proteins.

In a fifth embodiment, PLAP is used to reverse tumor induced hypoglycemia, a likely marker of early loss of skeletal muscle proteins.

In a sixth embodiment, the treatment regimen that is composed of placental alkaline phosphatase and chemotherapy also include IGF-1 to further enhance proliferation and survival of myoblasts.

In all the above embodiments, PLAP is used as an adjuvant enabling the actual chemotherapy to become more effective while at the same time effectively reduce chemotherapy induced loss of muscle proteins. While in the invention cyclophosphamide (Cyp) was used as the major model chemotherapeutic drug with known anticancer effects, in the clinical practice the invention allows to use PLAP together with any chemotherapeutic drug and their combinations as well as antibodies targeted against specific antigens on the surface of cancer cells.

### DETAILED DESCRIPTION

### 1. Definitions.

In this disclosure, the term “PLAP” refers to highly purified native placental alkaline phosphatase derived from human placenta via conventional purification methods. The term “rPLAP” refers to wild type recombinant PLAP with full catalytic activity. In the rPLAP protein, the 27-amino acid long glycosylphosphatidylinositol (GPI) anchor (present in purified native PLAP) is replaced with an 8-amino acid long FLAG sequence at the carboxyl terminal. This is a significant modification of the native PLAP. Since in the invention rPLAP reproduces the biological effects of PLAP, it is considered as an active derivative of PLAP. Based on this finding, it is reasonable to suggest that other active derivatives of PLAP may also be prepared. Other active derivatives of PLAP may also be recombinant hybrid alkaline phosphatases containing at least 50% portion of PLAP provided it has alkaline phosphatase activity and it detectably and in a statistically significant manner reduces loss of muscle proteins in chemotherapy treated tumor bearing subjects.

For simplicity, the term “PLAP” may also refer to other human alkaline phosphatases (APs) such as intestinal alkaline phosphatase (IALP), tissue-nonspecific alkaline phosphatase (TNAP), and germ cell (GCAP) as well as bovine intestinal alkaline phosphatase protein, purified or recombinant, in their glycosylated and non-glycosylated forms as well as peptides derived from these alkaline phosphatase proteins via conventional or recombination methods, that are effective in a statistically significant manner to at least partially reduce loss of muscle proteins in chemotherapy treated tumor bearing subjects. In this disclosure, the term “AP” means alkaline phosphatase when there is no need for further specification.

The term “highly purified” means a preparation of PLAP prepared from human placenta or another tissue (in case of other APs) or produced by a recombinant method which contains less than 1% of contaminant proteins by using a standard sodium dodecyl sulfate (SDS) gel electrophoresis protein separation method coupled with the commonly used Coomassie blue staining method and a densitometer for quantification of gel-bound stained proteins.

The term “therapeutically effective amount or dose” means a dose of PLAP that effectively, i.e. in a statistically significant manner, reduces the loss of skeletal muscle proteins in chemotherapy treated tumor bearing subjects.

In this invention, “chemotherapy” means any chemotherapeutic drug and their combinations, non-targeted or targeted, synthesized by a chemical method or produced by recombinant methods like antibodies targeted against specific antigens on the surface of cancer cells, that are used in the clinical practice to treat cancer.

In this invention, “early-stage loss of skeletal muscle proteins” means detectable tumor-and/or chemotherapy induced loss of skeletal muscle proteins when loss of body weight is less than 2% not yet reaching the threshold to define it as cachectic body weight loss.

Cachexia means, as defined by a panel of experts, (a) involuntary weight loss greater than 5% over the past 6 months, or (b) at BMI less than 20, weight loss greater than 2%, or (c) appendicular skeletal muscle index consistent with sarcopenia (males less than 7.26 kg/m²; females less than 5.45 kg/m²) accompanied by weight loss greater than 2%.

### 2. Placental Alkaline Phosphatase; the Active Component of the Invention.

Humans express four dimeric AP enzymes (E.C.3.1.3.1); the placental (PLAP), the intestinal (IALP), the tissue nonspecific (TNAP), and the germ cell (GCAP) enzymes, each catalyzing the hydrolysis of phosphomonoesters accompanied by the release of inorganic phosphate and alcohol [Millan, J.L. Alkaline phosphatases: Structure, substrate specificity and functional relatedness to other members of a large superfamily of enzymes. Purinergic Signalling, 2, 335-341; 2006; Kozlenkov, A., Manes, T., Hoylaerts, M.F. and Millan, J.L. Function assignment to conserved residues in mammalian alkaline phosphatases. J. Biol. Chem. 277, 22992-22999, 2002]. PLAP and GCAP are closely related (˜ 95% homology). The sequence homology between human IALP and PLAP is about 86.5% [Henthorn, P.S., Raducha, M., Edwards, Y.H., Weiss, M.J., Slaughter, C. and Harris, H. Nucleotide and amino acid sequences of human intestinal alkaline phosphatase: Close homology to placental alkaline phosphatase. Proc. Natl. Acad. Sci. U.S.A. 84, 1234-1238, 1987], and somewhat less between bovine (and calf) IALP and PLAP [ Weissig, H., Schildge, A., Hoylaerts, M.F., Iqbal, M. and Millan, J.L. Cloning and expression of the bovine intestinal alkaline phosphatase gene: biochemical characterization of the recombinant enzyme. Biochem. J. 290, 503-508, 1993]. TNAP is expressed in the bone, liver and kidney and is about 50% or more identical with the other three human APs. Various APs are expressed from bacteria to humans with the main features of enzyme's properties being conserved [Millan, J.L. Alkaline phosphatases: Structure, substrate specificity and functional relatedness to other members of a large superfamily of enzymes. Purinergic Signalling, 2, 335-341, 2006] strongly suggesting that other human and non-human APs may reproduce, at least in part, the biological effects of PLAP described in this invention.

PLAP may be produced in a recombinant form (rPLAP). The rPLAP used in this invention the 27-amino acid long glycosylphosphatidylinositol (GPI) anchor is replaced with a shorter FLAG sequence to ensure efficient secretion of the proteins from cells they are produced in. This is a significant modification of PLAP which provides excellent examples that the structure of PLAP may be significantly modified without losing its ability to stimulate cell proliferation in vitro and most probably in vivo as well. Accordingly, it is expected that some other recombinant forms of PLAP and other APs can be generated that will reproduce, at least in part, the positive effects of PLAP, as described in the Examples in details, on reducing the loss of skeletal muscle proteins and tumor growth. In this disclosure, highly purified native PLAP may be referred to as “PLAP” to distinguish it from the recombinant derivative of wild type PLAP (rPLAP).

Site-specific mutations may be introduced into PLAP thus creating new derivatives that do not alter the catalytic activity but cause changes in its membrane binding [Lowe, M.E. Site-specific mutations in the COOH-terminus of placental alkaline phosphatase: a single amino acid change converts a phosphatidylinositol-glycan-anchored protein to a secreted protein. J. Cell Biol. 116, 799-807, 1992].

It is also possible to create other shorter, but biologically still active, sequences (derivatives) of PLAP. For example, digestion of PLAP with the protease bromelain provided an active derivative which enhanced cell proliferation [Use of placental alkaline phosphatase to promote skin cell proliferation; U.S. Pat. No. 7,374,754, issued on May 20, 2008; Inventor, Zoltan Kiss]. These examples show that mutated or shortened forms of PLAP can be generated that retain, at least in part, the biological activity of the parent PLAP.

This invention can also use an alkaline phosphatase (AP) which is a hybrid derivative of two APs. For example, in such a hybrid one critical segment may originate from PLAP accounting for the heat stability, and another segment may originate from a different AP accounting for a higher catalytic phosphatase activity or another useful property. As an example, envisioned in the disclosure, such hybrid may contain up to roughly 50% of the sequence of PLAP providing stability, and up to roughly 50% of the sequence of another human AP providing the catalytic alkaline phosphatase activity. The rationale for using such hybrid is that of the APs, PLAP has by far the greatest stability in the circulation, while other APs may have greater catalytic activities towards some specific substrates. For example, this inventor found that ATP is a weak substrate of PLAP compared to IALP or TNAP (unpublished data).

Recombinant methods for obtaining appropriate preparations of PLAP and other APs are feasible. For example, using the cDNA of PLAP, recombinant protein may be produced by one of the many known methods for recombinant protein expression. PLAP has been cloned and expressed in different cell types [Kozlenkov, A., Manes, T., Hoylaerts, M.F. and Millan, J.L. Function assignment to conserved residues in mammalian alkaline phosphatases. J. Biol. Chem. 277, 22992-22999, 2002; Henthorn, P., Zervos, P., Raducha, M., Harris, H. and Kadesh, T. Expression of a human placental alkaline phosphatase gene in transfected cells: Use as a reporter for studies of gene expression. Proc. Natl. Acad. Sci. USA 85, 6342-6346, 1988; Chen, Y.H., Chang, T.C. and Chang, G.G. Functional expression, purification, and characterization of the extra stable human placental alkaline phosphatase in the Pichia pastoris system. Protein Expression & Purification 36, 90-99, 2004; Becerra-Artega, A., Mason, H.S. and Shuler, M.I. Production, secretion, and stability of human secreted alkaline phosphatase in tobacco NT1 cell suspension cultures. Biotechnol. Prog. 22, 1643-1649,

. Slightly modified forms of PLAP may be expressed in and obtained from other cell lines of human or animal origin, cow's milk, goat's milk, chicken egg, bacteria, and certain plant (for example, barley, rice, corn, wheat, tobacco) seeds or leaves.

If the recombinant PLAP, full length or active derivative, is derived from plants that are used for human consumption without restriction, a protein extract from such source may be used, after careful testing, for oral consumption without further purification of the protein. Any of the available suitable extraction methods known in the food industry can be used to produce such protein extracts from plants.

A preparation of human PLAP may be obtained by extraction from placental tissue. Human placenta synthesizes the enzyme during pregnancy, so that toward the end of the third term the level of PLAP in the placenta tissue and the maternal and fetal blood becomes high compared to other APs. Therefore, a preparation of PLAP may be obtained by butanol extraction of homogenized placenta. Other methods of extraction from placental tissue are also suitable. Tissue specific APs other than PLAP may also be extracted and purified from blood, liver, and other tissues of human or animal origins.

As used herein, the term “active PLAP” is used to refer to full length PLAP that significantly reduces or prevents tumor and/or chemotherapy induced loss of muscle proteins. As used herein, the term “active derivatives of PLAP” cover modified recombinant peptides containing sufficient lengths of the original sequence of PLAP that, at therapeutically effective amounts, effectively and in statistically significant manner reduce or prevent tumor and/or chemotherapy induced loss of muscle proteins.

The term “highly purified PLAP” is used herein to encompass preparations of PLAP that are obtained from human placenta by various purification steps. Highly purified PLAP contains less than 1% contaminating proteins that do not pose any significant health risk and do not reduce the beneficial effects of PLAP.

### 3. The Method of Treatment.

### 3/A. The Subjects Treated.

Subjects to be treated with PLAP as an adjuvant are male or female subjects of any age diagnosed with cancer and scheduled to receive chemotherapy. The primary purpose of added PLAP treatment is to prevent or reduce tumor and/or chemotherapy induced loss of muscle proteins thereby enhancing the efficacy of treatment on controlling tumor growth and eventually enhance life expectancy of cancer patients. The rationale for early PLAP treatment is that by reducing or preventing the loss of skeletal muscle proteins from the early stages of tumor development, further substantial loss of muscle proteins can be averted which translates into better control of tumor growth and increased life expectancy.

The half-life time of purified PLAP in human circulation is about 7 days [Clubb, J.S., Neale, F.C. and Posen, S. The behavior of infused human placental alkaline phosphatase in human subjects. J. Lab. & Clin. Med. 66, 493-507, 1965] and that of recombinant PLAP used in the invention is about 10 days (unpublished observation) which is much longer than the half-life time of other APs that can be between few hours and one day. This beneficial feature which allows less frequent applications (e.g., three times a week, twice a week, once a week and once every 10 days) makes PLAP the preferred AP to use as an adjuvant of chemotherapies.

While in this invention Cyp was used as the model anticancer compound, any other anticancer drugs used for chemotherapeutic treatments, alone or in combinations with other anticancer drugs, may be used. Examples for other prominent anticancer drugs (that do not represent the full list) that can be used together with PLAP include other platinum drugs (for example, Carboplatin, Oxaliplatin), Doxorubicin, Epirubicin, Altretamine, Azacitidine, Bleomycin, Busulfan, Capecitabine, Carmustine, Chlorambucil, Cladribine, Clofarabine, Cytarabine, Dacarbazine, Dactinomycin, Daunorubicin, Decitabine, Etoposide, Fluoroacyl, Fludarabine, Hydroxyurea, Idarubicin, Ifosfamide, Irinotecan, Mechlorethamine, Methotrexate, Melphalan, Mitomycin, Mercaptopurin, Mitoxantrone, Nelarabine, Paclitaxel, Docetaxel, Lomustin, Gemcitabine, Gemtuzumab ozogamicin, Mercaptopurine, Pentostatin, Procarbazine, Raltitrexed, Streptozocin (also named Streptozotocin), Teniposide, Thiotepa, Topotecan, or Valrubicin. In addition, PLAP may be used together with any targeted therapy, using monoclonal antibodies targeting antigens on the surface of cancer cells.

Most, if not all, chemotherapeutic agents are used in cycles allowing for the recovery of bone marrow function. However, PLAP is used three-times a week, twice a week, once a weck, or once every 10 days, as indicated by the clinical practice, regardless of the length of the cycles of chemotherapy.

### 3/B. Treatment via a Systemic Route.

In this invention, PLAP is administered to a human cancer patient by injection. Any suitable injection method, for example intravenous, subcutaneous, intraarterial, intramuscular, intraperitoneal, intraportal, or intradermal may be used. PLAP may also be administered via infusion or using an implanted device for controlled delivery.

For injection delivery, PL.AP may be dispersed in any physiologically acceptable carrier that does not cause an undesirable physiological effect and ensures proper distribution of PLAP into the desired area. Examples of suitable carriers include physiological saline and phosphate-buffered saline. PLAP may also be attached to nanoparticles and then dispersed in a suitable carrier. The injectable solution may be prepared by dissolving or dispersing a suitable preparation of PLAP in the carrier using conventional methods. As an example, a suitable composition for the practice in the method comprises PLAP in a 0.9% physiological salt solution to yield a total protein concentration of 1 mg/ml. Another suitable composition contains PLAP in a 0.9% physiological salt solution to yield a total protein concentration of 10 mg/ml. A third composition contains alkaline phosphatase in a 0.9% physiological salt solution to yield a total protein concentration of 50 mg/ml. As alternative methods, PLAP may be enclosed in liposomes, such as immunoliposomes, or attached to other delivery systems or formulations, such as, for example, nanoparticles or any other suitable carrier.

A suitable dosage for systemic administration may be calculated in grams of the active agent(s) per square meter of body surface area for the subject. In one embodiment, the therapeutically effective amount is between 0.005 to 2.5 g of PLAP per m² body surface of the mammal. In another embodiment, the therapeutically effective amount of PLAP is between 0.05 to 1 g per m² body surface of the mammal. In yet another embodiment, the therapeutically effective amount of PLAP is between 0.1 to 1 g per m² body surface of the mammal.

As for the timing of delivery, the therapeutically effective amount of PLAP may be administered three times a week, twice a week, once per week, or once in every 10 days.

Another factor to consider when determining the effective amount of PLAP is that it will be used as part of a more complex chemotherapy regimen with different characteristics and toxicity profile. As the general rule, if the toxicity of the chemotherapy is greater than that caused by average treatment, then PLAP may have to be used twice a week instead of once a week or once in every 10 days.

PLAP may be administered prior to, during, or after administration of chemotherapy. Other medication(s) or substance(s) used to treat a disease may be administered as prescribed without affecting the schedule of PLAP administration.

In embodiments of the invention, PLAP may be used together, in addition to the chemotherapy, with insulin like growth factor-1 (IGF-1) to further enhance the proliferation and/or survival of skeletal muscle cells (myoblasts and myotubes) in the skeletal muscle.

### EXAMPLES

### EXAMPLE 1. Purification and spectrophotometric assay of PLAP.

Human PLAP (Type XXIV, 1020 units of total activity) in a partially purified form was obtained commercially from Sigma-Aldrich. A butanol extraction of placental tissue performed by Sigma-Aldrich to obtain the partially purified material was followed by chromatography steps as described in [Chang, T .-C., Huang, S .-M, Huang, T .-M. and Chang, G.-G. Human placental alkaline phosphatase: An improved purification procedure and kinetic studies. Eur. J. Biochem. 209, 241-247, 1992] to obtain an essentially pure preparation of

PLAP as determined by sodium dodecyl sulfate-polyacrylamide gel electrophoresis (SDS-PAGE). The enzyme activity of PLAP was assayed using a spectroscopic method to monitor the hydrolysis of 4-nitrophenylphosphate (as an increase in absorbance at 410 nm) at room temperature (22° C.) as described in [Chang, G.-G., Shiao, M.-S., Lec, K.-R. and Wu, J.-J., Modification of human placental alkaline phosphatase by periodate-oxidized 1,N6-ethenoadenosine monophosphate. Biochem. J., 272, 683-690, 1990]. Activity analysis of 5-10-μg purified enzyme was performed in 1 mI. incubation volume containing 50 mM Na₂CO₃/NaHCO₃, 10 mM MgCl₂, 10 mM 4-nitrophenylphosphate at pII 9.8. The extinction coefficient of 4-nitrophenol was taken as 1.62×10⁴ M⁻¹ cm⁻¹. An enzyme activity of I U (unit) is defined as 1 umole substrate hydrolyzed/min at 22° C. at pH 9.8. The pure PLAP was further identified by sequence analysis performed by the Mayo Clinic Protein Core Facility (Rochester, MN, US). The specific activity of purified PLAP was 685 Units per mg protein.

### EXAMPLE 2. Production of Recombinant PLAP.

Wild type (with full catalytic activity) recombinant PLAP (rPLAP) was produced by a method described by others [Kozlenkov, A., Manes, T., Hoylaerts, M.F. and Millan, J.L. Function assignment to conserved residues in mammalian alkaline phosphatases. J. Biol. Chem. 277, 22992-22999, 2002]. In rPLAP the 27-amino acid long glycosylphosphatidylinositol (GPI) anchor (present in purified PLAP) is replaced with an 8-amino acid long FLAG sequence at the carboxyl terminal to ensure efficient secretion of the protein.

### EXAMPLE 3. Effects of subcutaneously administered purified PLAP in the cachectic PC-3 tumor model.

In this invention, mouse tumor models that lose significant body weight are called “cachectic”, while tumor models that do not lose significant body weight but lose muscle proteins are called non-cachectic. PC-3 human prostate cancer cell-derived aggressive tumors are cachectic depending on the mouse strain used for tumor development. In the following experiment, a cachectic mouse model of human prostate tumor was used.

PC-3 human prostate adenocarcinoma tissue pieces (0.1 cm³ volume) were implanted subcutaneously into the intrascapular region of 6 weeks old female mice [homozygous line of C.B .-171cr scid/scid] to develop the tumors. The mice were kept at specified pathogen free (SPF) hygienic levels. In the first group of mice (7 mice), no tumors were developed (tumor free control). In the second group of mice (7 mice), tumors were developed but remained untreated. In the third group of mice (7 mice), on days 8, 11, 14, 17, 21 and 25 after transplantation of PC-3 tumors, mice were administered 4 mg/kg of PLAP subcutaneously. The weight and total protein content of m. gastrocnemius (using a protein determination kit from Sigma), tumor weight, lean body weight (total weight minus tumor weight), and the weight of abdominal fat tissue were determined on day 28 (after tumor transplantation).

As shown in TABLE 1, PLAP treatment significantly reduced the loss of muscle proteins. PLAP also had the tendency to reduce the loss of muscle mass, but its effect did not reach the level of significance. PLAP clearly inhibited tumor growth (by 32%) as well reduced the loss of lean body weight (from 41% to 18%) and abdominal fat tissue (from 46% to 12%). This experiment demonstrates that if treatment with PLAP starts early during tumor development (8 days after tumor transplantation), it still can greatly reduce most of the key features of cachexia despite the presence of a fast-growing tumor. On the other hand, this experiment failed to clarify whether loss of body weight or loss of muscle proteins were the first to decline, or these changes evolved simultaneously.

TABLE 1
Summary of the effects of subcutaneously administered
PLAP in the cachectic PC-3 mouse tumor model.

| | Muscle | Muscle Protein | Tumor | | |
|---|---|---|---|---|---|
| | Mass | (mg/100 mg | Weight | Lean Body | Abdominal |
| Treatments (g) | (mg) | of muscle weight) | (g) | Weight (g) | Fat Tissue |
| Control (no tumor) | 98 ± 6 | 13.9 ± 1.2 | — | 29.3 ± 2.2 | 0.73 ± 0.09 |
| Untreated tumor | 79 ± 8 | 10.5 ± 0.7* | 4.2 ± 0.7 | 18.7 ± 2.6* | 0.40 ± 0.05* |
| PLAP treated tumor | 93 ± 9 | 13.3 ± 1.3** | 2.9 ± 0.4** | 25.9 ± 2.8** | 0.66 ± 0.07** |

*, **Significantly (P < 0.01-0.001) different from tumor-free and untreated tumors, respectively.

### EXAMPLE 4. Effects of recombinant rPLAP on the loss of muscle proteins, reduction of serum triglyceride, and hypoglycemia in the ‘non-cachectic’ human prostate tumor model.

It is not clear whether tumors can cause loss of skeletal muscle proteins in patients with stable body weight and no obvious reduction in muscle mass when the tumor is still small. In this experiment, it was tested (a) if a distinction among the onset of loss of muscle proteins, muscle mass, and body weight can be made using mice bearing human PC-3 tumor that did not seem to lose significant body weight during tumor development, and (b) if in mice that lost skeletal muscle proteins whether PLAP could reduce or prevent it as was seen above in the cachectic tumor model (TABLE 1).

In this experiment, 6 weeks old immune deficient NOD/SCID male mice (from Jackson Laboratory; code 005557; Nonobese Diabetic/SCID/II-2 gamma-null; NOD.Dg-Prkdescid Il2rg tm1wjl/SzJ), transplanted with 1.5 million PC-3 human prostate cancer cells on the back, were used. ['These mice are most often known by their branded name, NOD seid gamma (NSG™), They do not express the Prkdc gene nor the X-linked Il2rg gene. NSG mice are viable, fertile, normal in size and do not display any gross physical or behavioral abnormalities.]. The reason these mice were chosen because the transplanted tumors grow slowly that allowed separation of changes in muscle proteins, muscle mass/weight, and body weight.

Four groups of mice with 7 mice included in each group were used. The first group was tumor free control. In groups 2-4, PC-3 cells were transplanted on day 0, when mice were 6 weeks old. Mice in Group 2 remained untreated, while mice in Groups 3 and 4 were treated with intravenous injection of 1.0 mg/kg and 4.0 mg/kg body weight of recombinant PLAP (rPLAP), respectively, on days 11, 15, 18, 21, and 24 (days are counted from tumor transplantation).

Body weights were measured on days 0, 12, and 25. Tumor size was measured on day 25 followed by the last measurement of body weight and the removal of M. gastrocnemius. After measuring muscle weight, total proteins were determined by a “protein determination kit” (Sigma).

In each group, the total body weight slightly increased between day 12 and day 25 with no statistically significant differences among the 4 groups (TABLE 2). This experiment established that, (a) in the time frame of the experiment, these PC-3 tumor-bearing mice were not cachectic in the sense that they did not lose body weight, and (b) that rPLAP moderately inhibited tumor growth (like is the case with many other experimental tumors; not shown).

TABLE 3 shows that muscle weight was also not significantly changed within the detection limit. Despite the relatively small tumor size and no apparent changes in body weight and muscle weight, tumor bearing untreated mice lost about 12% of skeletal muscle proteins compared to the “No tumor; no treatment” group. rPLAP at both doses used reduced the loss of muscle proteins in a statistically significant manner (TABLE 3).

This experiment, repeated twice with similar results, leads to the important conclusion that at the early stages of tumor development when tumors are still small, there is already significant loss of skeletal muscle proteins but not of body weight or muscle weight. This implies that if the treatment of cancer patients with PLAP starts right after tumor detection when tumors are still small, there is a greater chance to prevent development of wasting of muscle proteins (and perhaps also prevent the loss of body weight) compared to starting the treatment later when cachexia (significant loss of body weight along with substantial loss of skeletal muscle proteins and muscle mass) already irreversibly took hold. This also means that by starting adjuvant PLAP treatment early, the efficacy of ongoing tumor treatments can be made more tolerable and efficient. In this invention, for practical purpose, the freshly diagnosed cancer patient is considered non-cachectic if he or she lost less than 2% of body weight during a 25-days period prior to cancer detection.

TABLE 2
Effects of intravenously administered recombinant PLAP
(rPLAP) in NOD/SCID male mice
with slowly growing PC-3 human prostate tumor.

| | Body Weight | | Tumor Weight |
|---|---|---|---|
| | (grams) | | (grams) |
| Tumor Treatment | Day 12 | Day 25 | Day 25 |
| No tumor; no treatment | 32.26 ± 1.46 | 34.03 ± 1.11 | — |
| Tumor; no treatment | 32.78 ± 0.89 | 33.41 ± 0.87 | 0.75 ± 0.38 |
| Tumor; rPLAP, 1.0 mg/kg | 32.21 ± 1.69 | 33.71 ± 1.71 | 0.53 ± 0.15 |
| Tumor; rPLAP, 4.0 mg/kg | 32.08 ± 1.24 | 34.00 ± 0.86 | 0.48 ± 0.31 |

*Significantly (P < 0.01) smaller than “No tumor; no treatment”.
**Significantly (P < 0.01) greater than “Tumor, no treatment”.

TABLE 3
Intravenously administered rPLAP reduced the loss of muscle
protein in the non-cachectic PC-3 tumor model.

| | Total Muscle Proteins | Muscle |
|---|---|---|
| | (mg/100 mg of | Weight |
| Tumor; Treatment | muscle weight) | (mg) |
| Tumor-free; no treatment | 13.65 ± 0.48 | 92 ± 7 |
| Tumor; no treatment | 11.83 ± 0.44* | 87 ± 5 |
| Tumor; rPLAP, 1.0 mg/kg | 12.75 ± 0.39** | 95 ± 10 |
| Tumor; rPLAP, 4.0 mg/kg | 13.22 ± 0.32** | 89 ± 6 |

*Significantly (P < 0.01) different from tumor free mice.
**Significantly (P < 0.01) different from untreated tumor.

In patients with cancer cachexia, triglyceride level in the blood is reduced, indicating increased catabolismby various lipase activities [Das, S.K. and Hoefler, G. The role of triglyceride lipases in cancer associated cachexia. Trends Mol. Med. 19, 292-301, 2013]. In the non-cachectic PC-3 tumor model (described to TABLE 3) as well, despite the small tumor size, there was a significant reduction in serum triglyceride level that was partially reversed by both doses of rPLAP as shown in TABLE 4. This experiment suggests that detecting decreased serum triglyceride levels can become a useful marker of the loss of muscle proteins at early stages of tumor development when there are no easily detectable changes in body weight and muscle weight. In the clinical practice, performing measurement of serum triglyceride is much easier, convenient, and less time consuming compared to measurement of muscle proteins for which there are usually no base values in the patient's medical record (unlike for serum triglyceride).

TABLE 4
rPLAP partially restored reduced serum triglyceride
level in the non-cachectic
PC-3 tumor model.

| | Serum Triglyceride |
|---|---|
| Tumor; Treatment | (mM) |
| Tumor-free; no treatment | 1.54 ± 0.17 |
| Tumor; no treatment | 0.83 ± 0.12* |
| Tumor; rPLAP, 1.0 mg/kg | 1.26 ± 0.18** |
| Tumor; rPLAP, 4.0 mg/kg | 1.32 ± 0.11** |

*Significantly (P < 0.01) different from tumor free mice.
**Significantly (P < 0.01) different from untreated tumor.

Tumor induced cachexia is occasionally accompanied by hypoglycemia. In the experiment described to TABLE 3, despite the small size of the tumor, blood glucose levels in untreated tumor bearing mice were lower than in tumor-free mice (TABLE 5). Treatment with rPLAP (last treatment was 24 hours prior to taking the blood samples at 0 min) corrected this mild hypoglycemia (see 0 min data in TABLE 5). After the administration of glucose (2 g/kg at 0 min), at each time point blood glucose levels in tumor bearing mice remained lower than in tumor free mice. In contrast, in rPLAP treated tumor bearing mice the blood glucose levels were closer to the values obtained with tumor free mice (TABLE 5). This data suggests that in cancer patients with no significant loss of body weight and muscle weight, hypoglycemia, along with reduced serum triglyceride, may be considered as another marker of the loss of skeletal muscle proteins. Importantly, PLAP treatment corrects tumor induced hypoglycemia.

TABLE 5
rPLAP normalized glucose tolerance in the PC-3 tumor model.

| | Blood Glucose (mM) | | | | |
|---|---|---|---|---|---|
| Tumor; Treatment | 0 min | 30 min | 60 min | 90 min | 120 min |
| Tumor-free; | 5.46 ± 0.43 | 14.08 ± 1.04 | 9.91 ± 0.60 | 7.82 ± 0.15 | 5.63 ± 0.37 |
| (no treatment) | | | | | |
| Tumor; | 4.36 ± 0.25* | 9.84 ± 0.69* | 7.85 ± 0.34* | 6.44 ± 0.70 | 4.55 ± 0.39* |
| (no treatment) | | | | | |
| Tumor; 1.0 mg/kg | 5.01 ± 0.23** | 12.54 ± 0.61** | 9.44 ± 0.26** | 7.80 ± 0.31** | 5.25 ± 0.40** |
| (rPLAP) | | | | | |
| Tumor; 4.0 mg/kg | 5.15 ± 0.16** | 13.95 ± 1.10** | 9.57 ± 0.20** | 7.75 ± 0.56** | 5.61 ± 0.26** |
| (rPLAP) | | | | | |

*Significantly (P < 0.01) different from tumor free.
**Significantly (P < 0.01) different from untreated tumor.

### EXAMPLE 5. Effects of PLAP in Cyp treated mice bearing T47D human estrogen receptor positive breast carcinoma.

T47D estrogen receptor positive human breast cancer cells were from American Tissue Culture Collection (10801 University Boulevard, Manassas, Virginia, 20110-2209, United States).

First generation hybrid BDF1 (C57BL female x DBA/2 male) 6 weeks old, weighing 22-23 grams, specified pathogen free (SPF) hygienic category colonies were used. The animals were kept in macrolon cage at 22-24 0 C° (45-55% humidity), with a lightning regimen of 12/12 h light/dark. The animals had free access to tap water and were fed with a sterilized standard diet (Charles River VRF1, autoclavable, Germany) ad libitum.

T47D mammary tumor pieces (maintained in mouse tail, containing about 1.5×10⁶ tumor cells) were implanted subcutaneously into the intrascapular region to develop the tumors. After 15 days, when the tumor-bearing mice were first treated, the sizes of the tumors were in the 0.27-0.34 cm³ range. PLAP and Cyp were dissolved in 0.9% sodium chloride and applied subcutaneously (PLAP) or intraperitoneally (Cyp) in 50 μl volume. In the first group (n=7), animals received no treatment. In the second group (n=7), mice were administered 120-mg/kg of Cyp on day 15 followed by eleven consecutive treatments on every second day (days 17, 19, 21, 23, 25, 27, 29, 31, 35, 37, and 39) with the same dose. In the third group (n=7), treatment with Cyp (on the same days as in group 2) was accompanied by administration of 7.5 mg per kg of PLAP on days 15, 20, 25, 30, 35, and 40. Tumor volume was measured on days 15, 18, 21, 24, 27, 31, 35, 40, and 46, and body weight was measured on day 46. Then the animals were kept without additional treatment to determine survival rates.

The body weights in the Cyp treated and Cyp+PLAP treated mice were 1.4 and 0.3% less, respectively, compared to the untreated group. As shown in Table 6 (numbers in parentheses indicate the number of mice alive), Cyp effectively inhibited tumor growth and from day 21 PLAP enhanced the effects of Cyp. Already data in TABLE 6 suggested that PLAP treatment extended life expectancy because on day 46 all mice survived in the “Cyp+PLAP” group, while 2 mice died in the “Cyp alone” group. Indeed, as shown in TABLE 7, PLAP significantly increased life expectancy of Cyp treated T47D tumor bearing mice. Cyp increased average survival from 38 days to 49 days and in combination with PLAP up to nearly 70 days (TABLE 7). This experiment indicates that PLAP can enhance life expectancy of Cyp treated tumor bearing mammals, and by extension that of human cancer patients treated with Cyp containing chemotherapy or with chemotherapy in general.

TABLE 6
Effects of PLAP on the growth of Cyp treated T47D tumors.

| | Tumor volume (cm³) on days after transplantation | | | | |
|---|---|---|---|---|---|
| Treatment | Day 15 | Day 18 | Day 21 | Day 24 | Day 27 |
| None | 0.34 ± 0.10 | 0.39 ± 0.16 | 1.04 ± 0.19 | 1.63 ± 0.39 | 2.28 ± 0.48 |
| Cyp | 0.30 ± 0.11 | 0.33 ± 0.11 | 0.56 ± 0.10 | 0.72 ± 0.20 | 0.91 ± 0.22 |
| Cyp + PLAP | 0.27 ± 0.08 | 0.38 ± 0.08 | 0.45 ± 0.10 | 0.59 ± 0.13 | 0.73 ± 0.11 |
| | Day 31 | Day 35 | Day 40 | Day 46 | |
| None | 3.30 ± 0.39 (6) | 4.57 ± 0.58 (4) | 6.18 ± 0.63 (3) | (0) | |
| Cyp | 1.06 ± 0.17 | 1.22 ± 0.32 (7) | 1.68 ± 0.29 (7) | 1.82 ± 0.24 (5) | |
| Cyp + PLAP | 0.84 ± 0.14 | 0.87 ± 0.21 (7) | 1.17 ± 0.16 (7) | 1.30 ± 0.22 (7) | |

TABLE 7
Effects of PLAP on the survival of
Cyp-treated T47D tumor bearing animals

| | Survival (days) | | |
|---|---|---|---|
| Treatment | Individual | Mean | % |
| None | 30, 34, 34, 37, 42, 44, 46 | 38.1 | 100 |
| Cyp | 44, 45, 49, 50, 50, 52, 55 | 49.3 | 129 |
| Cyp + PLAP | 60, 66, 67, 70, 75, 75, 76 | 69.8 | 183 |

### EXAMPLE 6. Effects of PLAP on skeletal muscle proteins in the Cyp treated human T47D tumor model.

Next, the T47 human breast cancer model described in EXAMPLE 5 were used to determine the effects of the tumor, Cyp, and PLAP on skeletal muscle proteins.

After 15 days of tumor implantation, when the tumor-bearing mice were first treated, the sizes of the tumors were in the 0.26-0.33 cm³ range. PLAP and Cyp were dissolved in 0.9% sodium chloride and applied subcutaneously (PLAP) or intraperitoneally (Cyp) in 50 ml volume. In the third and fifth groups, mice were administered 7.5 mg per kg of PLAP on of days 14, 17, 20, 23, and 26. In the fourth and fifth groups mice also received Cyp on days 15, 17, 19, 21, 23, 25, and 27. On day 28, body weights were measured first followed by determining the tumor volumes. After the mice were put to death under anesthesia, the gastrocnemius muscles were excised to determine total proteins by using a Sigma kit.

As shown in TABLE 8, the T47D tumor alone caused relatively small, but significant, decrease in skeletal muscle proteins that was further enhanced by Cyp treatment. PLAP significantly reduced both tumor and Cyp induced loss of muscle proteins. This experiment demonstrates that chemotherapy induced loss of skeletal muscle proteins can also be significantly reduced by PLAP.

TABLE 8
PLAP significantly reduced the loss of muscle proteins caused by
either the T47 tumor itself or the tumor and Cyp in combination.

| | Total Muscle Protein |
|---|---|
| Tumor; Treatment | (mg per 100 mg of muscle weight) |
| Tumor-free; no treatment | 13.44 ± 0.30 |
| Tumor; no treatment | 12.42 ± 0.47* |
| Tumor + Cyp | 11.31 ± 0.43** |
| Tumor + PLAP | 13.20 ± 0.38*** |
| Tumor + Cyp + PLAP | 13.07 ± 0.54*** |

*Significantly (P < 0.01) smaller than “Tumor-free, no treatment”.
**Significantly (P < 0.01) smaller than “Tumor, no treatment”.
***Significantly (P < 0.01) greater than “Tumor, no treatment, or Tumor, Cyp”.

### EXAMPLE 7. Effects of rPLAP, and PLAP in combination with IGF-1 on the proliferation of L6 myoblasts.

L6 myoblasts were grown in 96-well plates in 10% FBS containing DMEM up to 30% confluency. Then the medium was changed to serum-free medium, and after 2 hours, cells were treated with IGF-1 (50 nM), or rPLAP (150 nM), or PLAP (150 nM) in the absence or presence of IGF-1 for 72 hours, followed by the MTT assay (absorbance; A₅₄₀) as described by others assay [Carmichael, J, De Graff, W.G., Gazdar, A.F., Minna, J.D. and Mitchell, J.B. Evaluation of tetrazolium-based semiautomated colorimetric assay: Assessment of chemosensitivity testing. Cancer Res. 47, 936-942, 1987]. The data in TABLE 9 shows that rPLAP and PLAP had similar stimulatory effects on the proliferation of L6 myoblasts and they similarly enhanced the stimulatory effect of IGF-1. This data strongly suggests that rPLAP and PLAP can be considered as equivalent or nearly equivalent in achieving effects in the skeletal muscle and that they may be doing so in concert with IGF-1.

TABLE 9
PLAP and rPLAP similarly promote proliferation and survival
of L6 myoblasts both in the absence and presence of IGF-1.

| | L6 myoblast proliferation (A₅₄₀) |
|---|---|
| Treatment | (minus 0-time value) |
| Control, 72 hours | 0.137 ± 0.042 |
| IGF-1 | 0.280 ± 0.030* |
| PLAP | 0.267 ± 0.025* |
| PLAP + IGF-1 | 0.351 ± 0.033** |
| rPLAP | 0.255 ± 0.045* |
| rPLAP + IGF-1 | 0.332 ± 0.037** |

*Significantly (P < 0.01-0.001) different from the 72-hour control;
**Significantly (P < 0.01) different from IGF-1 alone.

### EXAMPLE 8. PLAP prevented the inhibitory effect of Myostatin on the proliferation of L6 myoblasts.

In this experiment, L6 myoblasts were seeded in 10% serum containing Dulbecco's modified Eagles (DMEM) medium in 96-well plates (n=8 for each treatment). After 24 hours at 30% confluence, the medium was changed to 2% serum-containing medium (0 time) and then treated with 0.25 μg/ml or 1 ug/ml myostatin (MSTN-from the R&D Systems-catalog no. 788-GB-010) in the absence or presence of 25 nM, 100 nM, or 200 nM PLAP for 96 hours. The relative number of viable cells (A₅₄₀) was determined by the MTT assay [Carmichael, J, De Graff, W.G., Gazdar, A.F., Minna, J.D. and Mitchell, J.B. Evaluation of tetrazolium-based semiautomated colorimetric assay: Assessment of chemosensitivity testing. Cancer Res. 47, 936-942, 1987]. The value measured at 0 time is subtracted from the 96-hour values.

The data in TABLE 10 (expressed as mean values +std. dev. of 8 determinations) indicates that 100-200 nM PLAP practically prevented the inhibitory effects of myostatin on the proliferation of L6 myoblasts.

IGF-1 is the only recognized know growth factor capable of preventing the negative effect of myostatin on the proliferation of myoblasts [Gehmert, S., Wenzel, C., Loibl, M., et. Adipose tissue-derived stem cell secreted IGF-1 protects myoblasts from the negative effects of myostatin. BioMed Res. International, Vol. 2014, Article ID 129048]. Myoblasts also express and release IGF-1. Data in TABLE 9 and TABLE 10, when considered together with the effect of IGF-1 on myostatin, suggests that PLAP and endogenous IGF-1 may act in concert on L6 myoblast proliferation and in preventing the negative effect of myostatin.

TABLE 10
PLAP prevented the inhibitory effects of Myostatin
on the proliferation of L6 myoblasts.

| | L6 myoblast proliferation minus | | |
|---|---|---|---|
| | 0-time value (A₅₄₀) | | |
| | | Myostatin | Myostatin |
| PLAP addition | No Myostatin | (0.25 μg/ml) | (1.0 μg/ml) |
| 96-hour control, | 0.340 ± 0.016 | 0.214 ± 0.032* | 0.175 ± 0.030* |
| no PLAP | | | |
| 96 hours, PLAP | 0.410 ± 0.025 | 0.325 ± 0.038** | 0.300 ± 0.023** |
| 25 nM | | | |
| 96 hours, PLAP | 0.541 ± 0.022 | 0.430 ± 0.024** | 0.402 ± 0.027** |
| 100 nM | | | |
| 96 hours, PLAP | 0.707 ± 0.032 | 0.542 ± 0.069** | 0.500 ± 0.033** |
| 200 nM | | | |

*Significantly (P < 0.01) lower than the 96-hour control value,
**Significantly (P < 0.01) higher than the corresponding myostatin value without PLAP.

Claims:

1. Administering human placental alkaline phosphatase or an active derivative thereof to a cancer patient without cachexia during chemotherapy, to reduce or prevent the loss of skeletal muscle proteins caused by tumor growth, or chemotherapy, or both.

2. The method of claim 1, wherein human placental alkaline phosphatase or an active derivative thereof is administered during chemotherapy to partially prevent reduction in serum triglyceride, a marker of early loss of skeletal muscle proteins.

3. The method of claim 1, wherein human placental alkaline phosphatase or an active derivative thereof is administered during chemotherapy to prevent hypoglycemia, a potential marker of early loss of skeletal muscle proteins.

4. The method of claim 1, wherein human placental alkaline phosphatase or an active derivative thereof is administered during chemotherapy to enhance the efficacy of chemotherapy.

5. The method of claim 1, wherein human placental alkaline phosphatase or an active derivative thereof is administered during chemotherapy to increase life expectancy.

6. The method of any one of claims 1-5, wherein the human alkaline phosphatase is isolated and highly purified from human placenta.

7. The method of any one of claims 1-5, wherein an active derivative of placental alkaline phosphatase, produced by cleaving placental alkaline phosphatase with a protease or is synthesized by a chemical method, is used.

8. The method of any one of claims 1-5, wherein the human placental alkaline phosphatase or an active derivative thereof is a recombinant protein, produced by a mammalian cell, a bacterium, a yeast, or purified from cow's milk, goat's milk, chicken egg, or a plant, which reproduces the effects of native placenta-derived placenta alkaline phosphatase on skeletal muscle proteins during chemotherapy.

9. The method of claim 8, wherein the recombinant protein is an active derivative of placental alkaline phosphatase having altered sequence(s) compared to a full length placental alkaline phosphatase which reproduces the effects of native placenta-derived placenta alkaline phosphatase on skeletal muscle proteins during chemotherapy.

10. The method of claim 8, wherein the recombinant placental alkaline phosphatase is a hybrid protein containing up to about 50% of an amino acid sequence of placental alkaline phosphatase sufficiently long to enhance stability in the circulation, and up to about 50% of an amino acid sequence of another alkaline phosphatase sufficiently long to provide catalytic alkaline phosphatase activity.

11. The method of claim 1, wherein the placental alkaline phosphatase or an active derivative thereof is administered to the cancer patient via injection in a physiologically acceptable carrier, such as 0.9% sodium chloride solution, liposomes, immune liposomes, nanoparticles, or any other suitable carrier, three times a week, twice a week, once a week, or once every 10 days.

12. The method of claim 11, wherein the injection is intravenous, intra-arterial, intraperitoneal, intramuscular, intraportal, subcutaneous, or intradermal.

13. The method of claim 11, wherein the placental alkaline phosphatase or an active derivative thereof is administered via infusion or by a controlled release device.

14. The method of any one of claims 11-13, wherein the placental alkaline phosphatase or an active derivative thereof is administered in therapeutically effective amounts in a range of between 0.005 to 2.5 g per m², or between 0.05 to 1 g per m², or between 0.1 to 1 g per m² of the calculated body surface of the cancer patient.

15. The method of any one of claims 11-14, wherein the placental alkaline phosphatase or an active derivative thereof is administered by one or more systemic routes prior to, during, or after chemotherapy.

16. The method of claim 1, wherein a treatment regimen, composed of placental alkaline phosphatase and chemotherapy also includes administering insulin like growth factor-1 to further enhance proliferation and survival of myoblasts.

17. The method of claim 1, wherein placental alkaline phosphatase or an active derivative is administered continuously as needed during chemotherapy.
